(19) United States
(12) Patent Application Publication
(10) Pub. No.: US 2007/0065420 A1
(43) Pub. Date: Mar. 22, 2007

(54) Ultrasound Therapy Resulting in Bone Marrow Rejuvenation

(76) Inventor: Lanny L. Johnson, Okemos, MI (US)

Correspondence Address: WILSON DANIEL SWAYZE, JR., 3804 CLEARWATER CT., PLANO, TX, 75025, US

(21) Appl. No.: 11/557,760

(22) Filed: Nov. 8, 2006

Related U.S. Application Data
(63) Continuation-in-part of application No. 11/210,078, filed on Aug. 23, 2005.

(51) Int. Cl. A61K 35/14; A61H 1/02

(52) U.S. Cl. 424/93.7; 601/1

(57) ABSTRACT

A method and system for treating a patient to repair damaged tissue which includes exposing a selected area of bone marrow of a patient to ultrasound waves or ultra shock waves so that cells comprising stem cells, progenitor cells or macrophages are generated in the area of the bone marrow of the patient due to the ultrasound, converting the cells from the bone marrow of the patient and reducing the damaged tissue in the bone marrow of the patient by repairing the damaged tissue.

### CROSS-REFERENCE TO RELATED APPLICATIONS

This application claims priority to Provisional Application Serial No. 60/607,676 filed Sep. 7, 2004 and is a continuation application in part of a non-provisional application, Ser. No. 11/210,078 and filed on Aug. 23, 2005.

### STATEMENT REGARDING FEDERALLY SPONSORED RESEARCH OR DEVELOPMENT

Not Applicable

### BACKGROUND OF THE INVENTION

(1) Field of the Invention

The present invention relates generally to mobilization of progenitor cells, stem cells and macrophages from bone marrow, and more particularly to mobilization by means of an internal physical treatment of the body. Specifically, the present invention relates to the mobilization of progenitor cells, stem cells and macrophages from bone marrow concomitant with bone surgery, diagnostic or treatment procedures utilizing such means as ultrasound, ultrasound shockwaves, surgical implantation, pulsed electromagnetic field (PEMF) therapy, CAT scans and magnetic resonance imaging (MRI) The present invention also relates to a device for the harvesting of marrow tissue during bone surgery.

(2) Description of the Related Art

Hematopoietic stem cells (HSCs) are cells which are capable of dividing and differentiating into any cell type of the blood. Two types of HSCs are known to exist: long-term and short-term HSCs. Long-term HSCs cell cycle and divide each day, while short-term HSCs differentiate into lymphoid and myeloid precursors. The lymphoid precursors give rise to T cells, B cells and natural killer cells. The myeloid precursors give rise to monocytes, macrophages, neutrophils, eosinophils, basophils, megakaryocytes, and erythrocytes. Hematopoietic stem and progenitor cells harvested for transplantation have typically come from bone marrow. Recently however, peripheral blood and umbilical cord blood have been used as a source for these cells. Peripheral blood stem cells (PBSC) have been mobilized by various techniques, since stem and progenitor cells are a very low percentage of the cells found in peripheral blood. An apheresis device is used to collect from a patient and automatically separate specific cells from whole blood. Afterwards the remaining blood components are returned to the patient, typically using a dual lumen catheter. Plasma, red blood cells, platelets, and white blood cells can be specifically removed by centrifugation in continuous mode while the remaining blood components are returned to the patient. Blood components which can be separated include plasma (plasmapheresis), platelets (plateletpheresis), macrophages and leukocytes (leukapheresis). Apheresis can be used to separate mononuclear cells (MNC) which include stem cells.

The stem and progenitor cells in peripheral blood can be increased prior to apheresis by myelosuppressive chemotherapy mobilization techniques and other drugs. Various myelosuppressive regimens are available including cyclophosphamide. Unfortunately, using such chemotherapy to mobilize PBSC can have an associated risk of toxicity. Since not every patient who must receive stem and progenitor cells will require chemotherapy for an associated illness, this is not always an appropriate option for mobilizing stem and progenitor cells from bone marrow. Recent approaches to mobilizing stem and progenitor cells utilize hematopoietic growth factors. Such growth factor mobilization procedures use filgrastim (granulocyte colony-stimulating factor [G-CSF]), sargramostim (granulocyte-macrophage colony-stimulating factor [GM-CSF]), or combinations thereof. G-CSF is administered subcutaneously at a dose of 10 to 16 micrograms per day (μg/d), which typically results in a peak level of circulating progenitor cells at day 4 to 7 after starting G-CSF administration. Other growth factors such as stem cell factor (SCF) can also be used to mobilize stem cells into the peripheral blood. Stress, injury, estrogen therapy, physical training, and nanopulses have all been shown to mobilize cells progenitor and stem cells in the peripheral blood.

Extracorporeal shockwave lithotripsy (ESWL) procedures are have been used to pulverize renal and ureteral calculi since 1980 and gallstones since 1985 into small fragments by utilizing shockwaves generated by a shockwave lithotripsy device. Additionally, stones have been treated in the common bile duct, pancreatic duct, and salivary glands. The shockwave lithotripsy devices have a shockwave generator component, a focusing system, a localization system, and a coupling means to transmit the shockwave energy to the patient. Three power sources for generating shock waves include electrohydraulic, piezoelectric or electromagnetic energy. In electrohydraulic (spark gap) devices a shock wave is initiated by an electric spark between electrodes at a first focal point of an ellipsoid and is focused to the second focal point of the ellipsoid inside of the patient. Piezoelectric devices direct the shock wave towards a focal point from an array of piezoelectric crystals mounted on a hemispherical dome. Electromagnetic devices generate a shock wave by a high current pulse in a coil to generate a magnetic field which drives a metal membrane to create the shock wave focused into the patient.

U.S. Pat. No. 4,905,671 to Senge et al. teach a method of bone growth induction using acoustic shock waves to the location where bone growth is desired, the shock waves producing bleeding at the site. U.S. Pat. No. 5,393,296 to Rattner et al. teach a method for the stimulation of bone growth using acoustic rarefaction pulses to a bone where bone growth is to occur which produces hemorrhage, microfissures and at least partially loosened bone chips. U.S. Pat. No. 5,520,612 to Winder et al. teach a method of using low-frequency acoustic energy to accelerate repair of bone fracture with an ultrahigh acoustic carrier frequency applied adjacent to the fracture space which acts as a wave guide to establish a vibrating standing-wave within the fracture. U.S. Pat. No. 5,595,178 to Voss et al. teach a method of exposing a patient to acoustic shock waves to treat changes in human or animal bones which cause a boundary surface gap with a width of less than five millimeters to form between the bone and an acoustically reflective body such as an implant, a tooth or a bone fragment. Vibrations are generated in the bone surfaces, the surfaces of the acoustically reflecting body, and at the gap by multiple reflections of the generated shock waves.

U.S. Pat. No. 6,390,995 to Ogden et al. teaches a method of applying acoustic shock waves to a site of a pathological condition to induce micro-injury and increase vascularization so as to accelerate healing at the site.

U.S. Patent Application Publication No. 2004/0049134 to Tosaya et al. teach the therapeutic treatment of brain-plaques, fibrils, abnormal-protein related or aggregation-prone protein related deposition-diseases employing acoustic energy applied to a region of the brain. The therapy results in: (i) physical breakup of the deposits, (ii) interference in at least one deposit formation process, or (iii) aiding the recovery, growth, regrowth or improved functionality of brain-related cells or functional pathways impacted by the deposits, or supporting the growth of newly transplanted cells anywhere in the brain-related anatomy to treat Alzheimer's and other deposition-related disorders of the brain.

While the related art teach various internal physical means of diagnosis and treatment of the body, and the related art teach chemical or hematopoietic growth factor mobilization of stem and progenitor cells from bone marrow, there still exists a need for methods of mobilizing cells from the bone marrow which can be harvested and introduced into tissues of a patient to repair and regenerate damaged tissue. There is a need also just to activate without harvest and to send to areas of disease or injury.

The bone marrow is the source of pleuripotential stem cells that have healing potential in case of injury or disease. The bone marrow is also the home of the hematopoietic system, thereby manufacturing red and white blood cells to the body with the attendant immune system components.

The bone marrow becomes less cellular and less vascular with age which is termed conversion. There is need for therapeutic measures to restore bone marrow due to normal aging and/or due to disease or injury to a more vital youthful health and healing potential status. This is termed reconversion of bone marrow and occurs physiologically and under various pathological conditions.

Conversion of Bone Marrow: Normal changes in bone marrow occur with aging. This natural process has been named conversion. Changes in normal bone marrow converts from cellular to fatty marrow in a predictable pattern and is usually completed by age 18-25 years. It is gradually converted from predominately red to yellow, from vascular and cellular to fatty in nature. This is easily and well documented by MRI.

The histologic study of bone marrow by Dunnill et al, in 1967, demonstrated that the volume of red marrow in vertebral bodies decreases from a mean of 58% in the 1st decade of life to a mean of 29% in the 8th decade of life. Dunnill M S, Anderson J A, Whitehead R. Quantitative histological studies on age changes in bone. J Pathol Bacteriol 1967; 94:275-291.

Concomitantly, there is an even greater increase in the percentage of fatty marrow with age. Ricci et al, in 1990, also demonstrated similar findings for fatty bone marrow distribution by using in vivo MR imaging. Ricci C, Cova M, Kang Y S, et al. Normal age-related patterns of cellular and fatty bone marrow distribution in the axial skeleton: MR imaging study. Radiology 1990; 177:83-88.

There is a distal to proximal conversion trend in the skeleton. The remaining areas of red marrow are the axial skeleton, the proximal humerus, the proximal femur. Older individuals commonly have the spine and pelvis dominated by yellow or fatty marrow.

The histomorphometric measurements performed by Demmler et al, in 1983, also demonstrated that reduced hematopoietic elements in bone marrow are accompanied by a corresponding increase in fat cells and a decrease in arterial capillary and sinus numbers. These pieces of evidence further support our finding that decreased bone marrow perfusion is associated with increased age and fatty marrow percentage. Demmler K, Otte P, Bartl R, et al. Osteopenia, marrow atrophy and capillary circulation: comparative studies of the human iliac crest and 1st lumbar vertebra. Z Orthop 1983; 121:223-227.

Reconversion: Reconversion is the process of changing back to red bone marrow seen in youth. It is the changing back of the bone marrow from fatty to red. When is occurs it happens in the reverse order of conversion, progressing from proximal to distal in the skeleton.

Physiologic Reconversion: Reconversion may be physiologic and reversible. It is seen in stress as when the marrow is stressed as with hypoxemia.

Pathological Reconversion: Stress results in reconversion. It has been seen in obese women who smoke and in heavy smokers. It has been identified in sleep apnea. It has been identified by MRI in various types of anemia and or infiltrative disease of certain malignancies. It may be seen in infection, leukemia, lymphoma, myeloma. It has been seen in sickle cell anemia, Thalassemia and early stage of Gaucher disease. MR shows decreased signal intensity (SI) on all the conventional sequences (T1, T2, STIR).

Post Traumatic Blast Localized Reconversion: Clinical evidence of localized mobilization of stem cells following high energy blast has been recently observed in war injuries with traumatic amputations. There is a great proliferation of bone at the amputation stump which complicates treatment and subsequent fitting of a prosthesis. This was published in USA Today with the following quote from expert in bone overgrowth. “High-intensity blasts, which can shred muscles, tendons and bone, appear to stimulate adult stem cells to heal the damage, says Vincent Pellegrini Jr., a professor and chairman of the orthopedics department at the University of Maryland School of Medicine.” Szabo L. Bone Condition hampers soldier's recovery. USA Today, Feb. 12, 2006.

Pharmacological Reconversion: Pharmacological reconversion has been reported following Granulocyte colony stimulating factor (GCSF) used to stimulate myeloid cell production in children undergoing chemotherapy for osteosarcoma. It has also been seen after growth factor administration with chemotherapy.

Tracking reconversion: MRI is thought to be more sensitive to presence of microscopic fat than anatomical data by histology. MRI is also valuable in tracking changes in marrow to measure the effect on a therapy.

Differential Diagnosis of Reconversion: Awareness of the various factor causing reconversion is important in clinical interpretation versus malignancy. Supermagnetic iron oxides are useful in differentiating the normal from neoplastic bone marrow.

### OBJECTS

Therefore, it is an object of the present invention to provide a method of treating a patient to regenerate damaged tissue.

It is further an object of the present invention to provide a method of mobilizing cells from the bone marrow utilizing physical means.

It is still further an object of the present invention to provide a device for the harvesting of bone marrow during bone surgery.

It is still a further object of the present invention to provide a system and method to repair the bone marrow of a patient.

It is a further object of the present invention to provide a system and method to increase the Cellularity of the bone marrow.

It is a further object the invention to provide a system and method to increase the vascularity of the bone marrow.

These and other objects will become increasingly apparent by reference to the following description.

### SUMMARY OF THE INVENTION

The present invention provides methods for the mobilization of stem cell, progenitor cells and/or macrophages from bone marrow, and more particularly to mobilization by means of an internal physical treatment of the body. Specifically, the present invention encompasses means to mobilize stem cells, progenitor cells and/or macrophages from bone marrow concomitant with bone surgery, diagnostic or treatment procedures utilizing such means as ultrasound, ultrasound shockwaves, surgical implantation, pulsed electromagnetic field (PEMF) therapy, CAT scan and magnetic resonance imaging (MRI).

The present invention provides a method for treating a patient to repair damaged tissue which comprises exposing a selected area of bone of a patient to ultrasound waves or ultra shock waves so that stem cells, progenitor cells and/or macrophages are released into the bloodstream of the patient from the area due to the ultrasound, harvesting the cells from the bloodstream of the patient, optionally culturing the cells, and introducing the cells to the damaged tissue of the patient so as to repair the damaged tissue.

In further embodiments of the method, the area comprises the bone in a trunk or extremity of the patient so that the cells are released from marrow of the bone. In further embodiments of the method, the cells are introduced to an organ as the damaged tissue. In still further embodiments of the method, the cells are introduced to cartilage as the damaged tissue. In still further embodiments of the method, the cells are introduced to bone as the damaged tissue. In still further embodiments of the method, the cells are introduced to bone marrow as the damaged tissue. In still further embodiments of the method, the patient is a human. In still further embodiments of the method, the patient is an animal. In still further embodiments of the method, the shock waves are from a lithotripsy apparatus which are directed into the area.

In still further embodiments of the method, the area is the bone in an extremity of the patient. In further embodiments of the method, the bone is in an arm or a leg. In further embodiments of the method, the the area is the bone in a trunk of the patient. In still further embodiments of the method, the bone is a sternum or an iliac crest.

The present invention provides a method for treating a patient to repair damaged tissue which comprises exposing a kidney stone in a patient to ultrasound waves or ultra shock waves so that stem cells, progenitor cells and/or macrophages are released into the bloodstream of the patient from the area due to the ultrasound, harvesting the cells from the bloodstream of the patient, optionally culturing the cells, and then introducing the cells into the damaged tissue of the patient so as to repair the damaged tissue.

The present invention provides a method for treating a recipient patient to repair damaged tissue which comprises exposing an area in a donor patient (i.e. pelvis, sternum and long bones) to ultrasound waves or ultra shock waves so that stem cells, progenitor cells and/or macrophages are released into the bloodstream of the donor patient from the area due to the ultrasound, harvesting the cells from the bloodstream of the donor patient, and introducing the cells into the damaged tissue of the recipient patient so as to repair the damaged tissue.

The present invention provides a system for harvesting stem cells, pluripotential cells or progenitor cells, and/or macrophages which comprises a container for a bath which provides ultrasound waves or shock waves to an area of an extremity of a patient immersed in the bath so as to generate cells selected from stem cells, pluripotent cells, progenitor cells, macrophages, and mixtures thereof in the bloodstream, harvesting means for removing the cells from the bloodstream.

In further embodiments, the system further comprising a fluid for submersing the extremity of the patient. In further embodiments of the system, the bath is for an arm or a leg.

The present invention provides a method for treating a patient to repair damaged tissue which comprises: providing a selected area of the patient to be exposed; and exposing the selected area of the patient to a physical treatment of the body selected from the group consisting of ultrasound waves, ultra shock waves, bone surgery, CAT scan and magnetic resonance imaging (MRI) so that stem cells, progenitor cells and/or macrophages are released into the bloodstream of the patient from the area due to the physical treatment and such that the stem cells or progenitor cells migrate to the damaged tissue of the patient so as to repair the damaged tissue. In further embodiments of the method the damaged tissue is a muscle. In still further embodiments the damaged tissue is a ligament. In still further embodiments the damaged tissue is a tendon. In still further embodiments the damaged tissue is a tendon, cartilage, heart, liver, nerve or spinal cord. In still further embodiments of any one of the methods, the cell is a fibroblast.

The present invention provides a method for providing a store of stem cells, progenitor cells and/or macrophages of a patient for future use which comprises: exposing a selected area of a patient to a physical treatment of the body selected from the group consisting of ultrasound waves, ultra shock waves, bone surgery, CAT scan and magnetic resonance imaging (MRI) so that stem cells, progenitor cells and/or macrophages are released into the bloodstream of the patient from the area due to the ultrasound; harvesting the cells from the bloodstream of the patient; and freezing the cells harvested from the bloodstream of the patient so as to provide a store of stem cells, progenitor cells and/or macrophages of the patient for future use.

The present invention provides a method for treating a patient to repair damaged tissue which comprises performing a surgical procedure upon a selected area of bone of a patient so that stem cells, progenitor cells and/or macrophages are released into the bloodstream of the patient from the area due to the surgical procedure, harvesting the cells from the patient, and introducing the cells to the damaged tissue of the patient so as to repair the damaged tissue. In further embodiments of the method the surgical procedure is total joint surgery. In still further embodiments of the method the surgical procedure is open reduction internal fixation (ORIF) of fractured bone. In further embodiments of the method the cells are harvested directly from marrow exposed during the surgical procedure. In still further embodiments of the method the cells are harvested from the bloodstream of the patient.

The present invention provides a method for treating a patient to repair damaged tissue which comprises: performing a surgical procedure upon a selected area of bone of a patient; harvesting bone marrow cells from the patient; isolating a population of cells from the bone marrow cells; and introducing isolated population of cells to the damaged tissue of the patient so as to repair the damaged tissue. In further embodiments of the method the surgical procedure is total joint surgery. In still further embodiments of the method the surgical procedure is open reduction internal fixation (ORIF). In still further embodiments of the method the isolated population of cells are stem cells, progenitor cells, macrophages or precursors of macrophages.

The present invention provides a method for treating a patient to repair damaged tissue which comprises exposing a patient to an external magnetic field and an applied oscillating electromagnetic field so that stem cells, progenitor cells and/or macrophages are released into the bloodstream of the patient due to the exposure, harvesting the cells from the patient, and introducing the cells to the damaged tissue of the patient so as to repair the damaged tissue. In further embodiments of the method the patient is exposed to the external magnetic field and the applied oscillating electromagnetic field during a magnetic resonance imaging (MRI) procedure.

The present invention encompasses an instrument used during surgery or separately for percutaneous harvest of cells by entering an end of a long bone, such as a femur. Therefore, the present invention provides a surgical instrument for collecting bone marrow tissue having a proximal end and an opposing distal end, the instrument comprising: (a.) a handle at the proximal end of the surgical instrument for gripping by a surgeon; (b.) an elongate hollow tube attached to the handle, the hollow tube having an opening at a first end of the hollow tube for attachment to a vacuum means, and a pointed tip at a second end of the hollow tube, the pointed tip situated at the distal end of the surgical instrument; (c.) one or more distal openings in the hollow tube adjacent to the tip which allow fat and cells to enter the tube when a suction is provided by the vacuum means connected to the open end of the hollow tube so as to draw out the marrow tissue from the bone for collection; and (d.) one or more secondary slits along the hollow tube which provide venting so as to avoid clogging of the one or more distal openings when the suction is provided by the vacuum means.

In further embodiments of the instrument the handle comprises: (a) a grip having a first end and a second end for gripping by the surgeon; and (b) a shaft attached to the hollow tube, wherein the grip is attached perpendicularly to the shaft equidistant between the first end and the second end so as to form a T-shape.

The present invention provides methods for the mobilization of stem cell, progenitor cells and/or macrophages from bone marrow, and more particularly to mobilization by an internal physical treatment of the body. Specifically, the present invention encompasses devices to mobilize stem cells, progenitor cells and/or macrophages from bone marrow to convert the bone marrow utilizing such devices as ultrasound, and ultrasound shockwaves.

The present invention provides a method for treating a patient to repair damaged tissue which comprises exposing a selected area of bone marrow of a patient to ultrasound waves or ultra shock waves so that stem cells, progenitor cells and/or macrophages are activated to convert the bone marrow of the area due to the ultrasound so as to repair the damaged tissue.

In further embodiments of the method, the area comprises the bone marrow in a trunk or extremity of the patient so that the cells are released within the marrow of the bone to convert the bone marrow by increasing the cellular of the bone marrow.

In further embodiments of the method, the fatty bone marrow is reduced. In further embodiments of the method, the vascular of the bone marrow is increased. In still further embodiments of the method, the patient is a human. In still further embodiments of the method, the patient is an animal. In still further embodiments of the method, the shock waves are from a lithotripsy apparatus which are directed into the area.

In still further embodiments of the method, the area is the bone marrow in an extremity of the patient. In further embodiments of the method, the bone marrow is in an arm or a leg. In further embodiments of the method, the area is the bone marrow in a trunk of the patient. In still further embodiments of the method, the bone marrow is a sternum or an iliac crest. In still further embodiments, the bone marrow is within the head of the patient. In still further embodiments, the bone marrow is within the back of the patient. In still further embodiments, the bone marrow is in the feet of the patient. In still further embodiments, the bone marrow is in the hands of the patient.

The present invention provides a method for treating a recipient patient to repair damaged tissue which comprises exposing an area in the bone marrow of a donor patient (i.e. pelvis, sternum and long bones) to ultrasound waves or ultra shock waves so that stem cells, progenitor cells and/or macrophages are released into the bone marrow of the donor patient due to the ultrasound, converting the bone marrow of the donor patient so as to repair the damaged bone marrow.

The present invention provides a system for activating stem cells, pluripotential cells or progenitor cells, and/or macrophages which comprises a container for a bath which provides ultrasound waves or shock waves to an area of an extremity of a patient immersed or adjacent in the bath so as to generate cells selected from stem cells, pluripotent cells, progenitor cells, macrophages, and mixtures thereof in the bone marrow, the ultrasound waves or ultrasound shock waves converting the bone marrow.

In further embodiments, the system further comprising a fluid for submersing the extremity of the patient. In further embodiments of the system, the bath is for an arm or a leg.

The present invention provides a method for treating a patient to repair damaged bone marrow which comprises: providing a selected area in the bone marrow of the patient to be exposed; and exposing the selected area in the bone marrow of the patient to a physical treatment of the body selected from the group consisting of ultrasound waves, and ultra shock waves so that stem cells, progenitor cells and/or macrophages are released in the bone marrow of the patient from the area due to the physical treatment and such that the stem cells or progenitor cells convert the damaged tissue of the patient so as to repair the damaged tissue.

In still further embodiments, the bone marrow is located in the extremities. In still further embodiments, the bone marrow is located in a leg. In still further embodiments the bone marrow is located in an arm. In still further embodiments, the bone marrow is located in a hip In still further embodiments the bone marrow is located in a rib. In still further embodiments, the bone marrow is located in a shoulder. In a still further embodiments, the bone marrow is located in an arm. In still further embodiment, the bone marrow is located in the hand. In still further embodiment, the bone marrow is located in the back. In still further embodiment, the bone marrow is located in the axial skeleton.

### BRIEF DESCRIPTION OF THE DRAWINGS

FIG. 1 shows a patient undergoing treatment according to one embodiment of the present invention.

FIG. 2 is a top view of an instrument 100 for harvesting bone marrow tissue.

FIG. 3 is a side view of the instrument 100 for harvesting bone marrow tissue.

FIG. 4 is a cross-section view of the instrument 100 for harvesting bone marrow tissue taken along line 4-4 of FIG. 3 showing distal openings 122.

FIG. 5 is a cross-section view of the instrument 100 for harvesting bone marrow tissue taken along line 5-5 of FIG. 3 showing secondary slits 120.

FIG. 6 illustrates a patient undergoing treatment in accordance with another embodiment of the invention;

FIG. 7 illustrates details of the device for providing treatment to the patient.

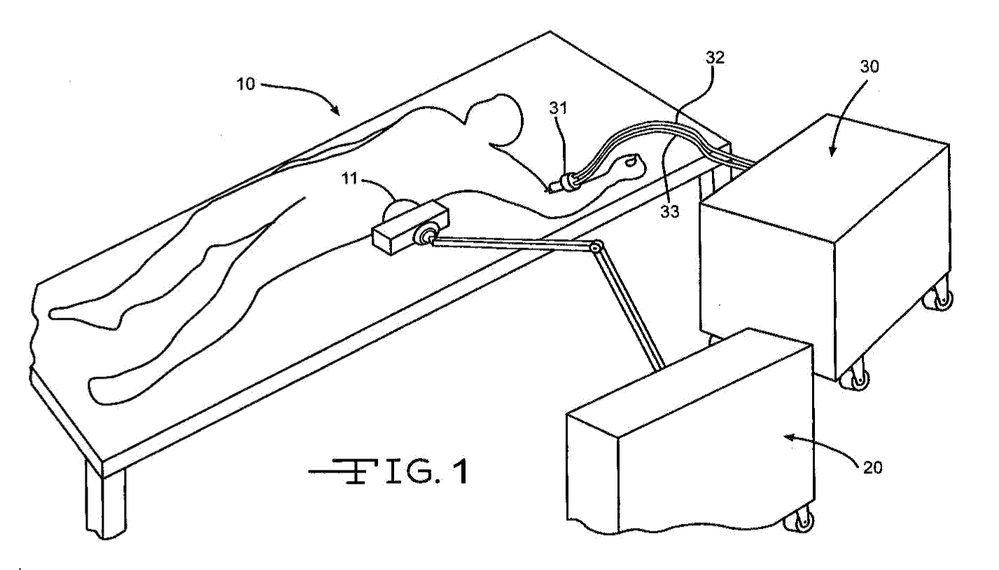

### DETAILED DESCRIPTION OF THE INVENTION

All patents, patent applications, government publications, government regulations, and literature references cited in this specification are hereby incorporated herein by reference in their entirety. In case of conflict, the present description, including definitions, will control.

The term “pluripotent” used herein refers to cells which have developmental plasticity and are capable of giving rise to cells derived from any of the three embryonic germ layers, including the mesoderm, endoderm, and ectoderm.

The term “stem cells” used herein refers to undifferentiated cells which are capable of dividing and self-renewal for extended periods, are unspecialized, and can differentiate into many lineages of specialized cell types.

The term “progenitor cells” used herein refers to unspecialized or partially specialized cells which differentiated from stem cells and which have the capacity to divide into more than one specialized cell types.

The risk of developing a disease that could require a stem cell transplant has been estimated to be as high as one in three hundered according to Bone & Marrow Transplant Newsletter. Stem cells and progenitor and multi-potential cells are known to reside within the various tissues of the body, including the bone marrow and small intestine. Stem cells from the bone marrow are harvested during the medical treatment of cancers and tissue engineering projects. It is known that during the placement of a total hip stem or intra-medullary rod that marrow fat and cells are forced into the blood stream, often in amounts that may cause an embolism. Lithotripsy is utilized in the present invention to affect the adjacent bone marrow and cause marrow cells to spill out into the circulating blood. In another embodiment of the present invention localized ultra sound to bone results in the same mobilization of marrow cells.

The present invention provides a method for treatment to repair damaged tissue which comprises mobilizing cells by physical means so that cells comprising stem cells, pluripotential cells or progenitor cells, macrophages, monocytes and/or other precursors of macrophages are released into the bloodstream of the patient from the area due to the physical means, harvesting the cells from the bloodstream of the patient, and introducing the cells to the damaged tissue of the patient or another patient so as to repair the damaged tissue. In some embodiments, the stem cells, pluripotential cells or progenitor cells, and/or macrophages can be frozen and stored by techniques known in the art to preserve the cells for future thawing and use if needed later in life. In some embodiments of the present invention, the damaged tissue which is treated is mesenchymal, such as bone, cartilage, muscle, ligaments, tendons, and bone marrow. In other embodiments, the damaged tissue which is treated is non-mesenchymal (i.e. endodermal or ectodermal tissue types). Mesenchymal stem cells can express phenotypic characteristics of be endothelial, neural, smooth muscle, skeletal myoblasts, and cardiac myocyte cells, therefore the present invention can be used to replace these cell types. In some embodiments of the present invention, the damaged tissue is spinal cord or other nerves of the central or peripheral nervous system. One example is the treatment of spinal cord injury by means of the present invention. Some findings indicate that bone marrow stem cells can differentiate into epithelial cells. Therefore, in some embodiments, the damaged tissues include liver, kidney, lung, skin, gastrointestinal tract. Other studies indicate that bone marrow stem cells can differentiate into myocytes. Therefore, in further embodiments the damaged tissues are heart or skeletal muscle. While some embodiments require the cells to be isolated and reintroduced into the damaged tissues, other embodiments encompassed by the present invention rely upon the ability of stem cells which have been mobilized to home in on the damaged tissue without the step of isolating the cells from the patient's blood. Furthermore, in some embodiments the treatment is allogeneic, that is, the stem cells from or progenitor cells are isolated from a donor patient and are used to treat damaged tissue in a recipient patient.

Mesenchymal stem cells are capable of differentiating into various cell types, including at least osteoblasts, chondrocytes, adipocytes, myoblasts, fibroblasts and marrow stroma. Chondrocytes synthesize randomly oriented type II collagen and proteoglycans as their extracellular matrix to thereby form cartilage. Osteoblasts form bone on cross-linked type I collagen in alternately parallel and orthogonal oriented laminae.

Physical Mobilization of Stem Cells, Progenitor Cells, and/or Macrophages: A variety of physical means can be used in the present invention to mobilize stem cells, progenitor cells, and/or macrophages. The cells can be retrieved incidental to or concomitant with a diagnostic modality or a treatment method. Some examples of physical means to mobilize cells encompassed by the present invention include bone surgery, magnetic resonance imaging, and ultrasound including diagnostic ultrasound and therapeutic shockwave treatments such as lithotripsy.

Mobilization via Bone Surgery: It is known in the art and reported in literature that marrow tissue including fat and cells are physically mobilized during total hip stem implantation into the femur. The marrow mobilization has been identified during surgery by ultrasound monitoring via the esophagus. The mass of marrow tissue transported to the heart and then to the lungs can even result in cardiac arrest. Furthermore, vascular transportation to the brain may result in a stroke syndrome.

The common means of harvesting bone marrow cells is by needle biopsy of the sternum or the pelvic bone iliac crest. This method yields small amounts of tissue. Other opportunities exist for bone marrow cell harvesting. The bone marrow is regularly exposed during routine surgery under sterile conditions. This is most common during total joint replacement and internal fixation of long bone fractures. The amount of marrow tissue potentially far exceeds that available by needle biopsy.

Amongst the harvesting methods proposed there is one unique surgical situation. It is well known that the sudden introduction of a cylindrical or other shaped instrument or prosthesis into the femur during total hip surgery mobilizes bone marrow tissue and fat into the blood stream. The avenue is through the bony cortex (outer shell) via the small perforating veins. This event probably occurs to some extent with every surgical maneuver of this type. The medical literature draws attention to this event as a probable cause of inter-operative complication of fat embolism to the heart, lungs and brain.

My unpublished laboratory experiments have confirmed this mechanism. It was further learned that the introduction of any instrument or device greater that 6 mm diameter into the proximal femoral shaft produced this phenomena. It was further observed that a hollow instrument less than 6 mm in diameter attached to suction would remove bone marrow tissue, predominately fat and cells. The subsequent placement of a instrument of larger diameter did not result in expulsion of marrow fat and cells out of the venules of the bony cortex. Thus preliminary preparation of the femoral canal in such a manner had potential to reduce such a complication.

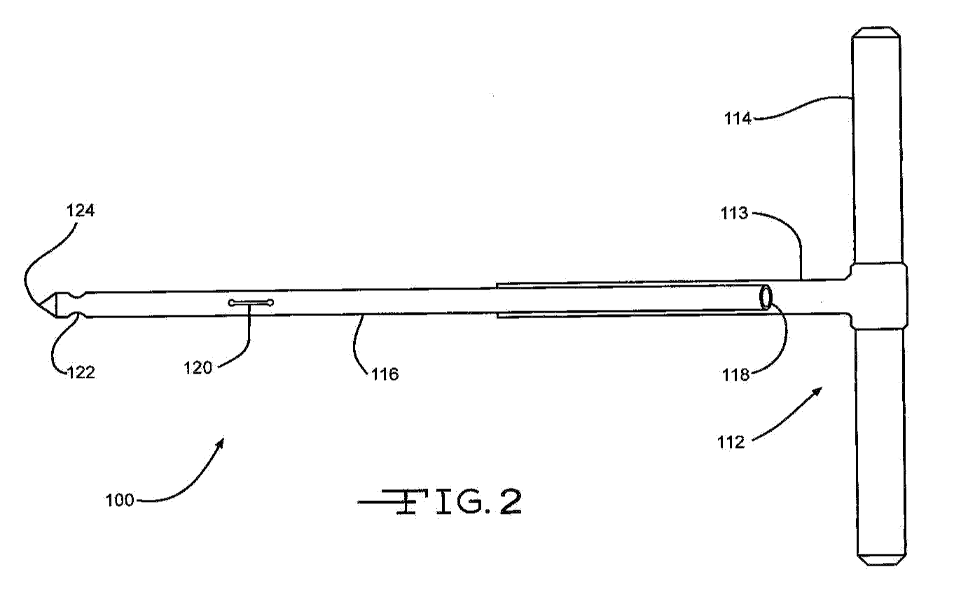

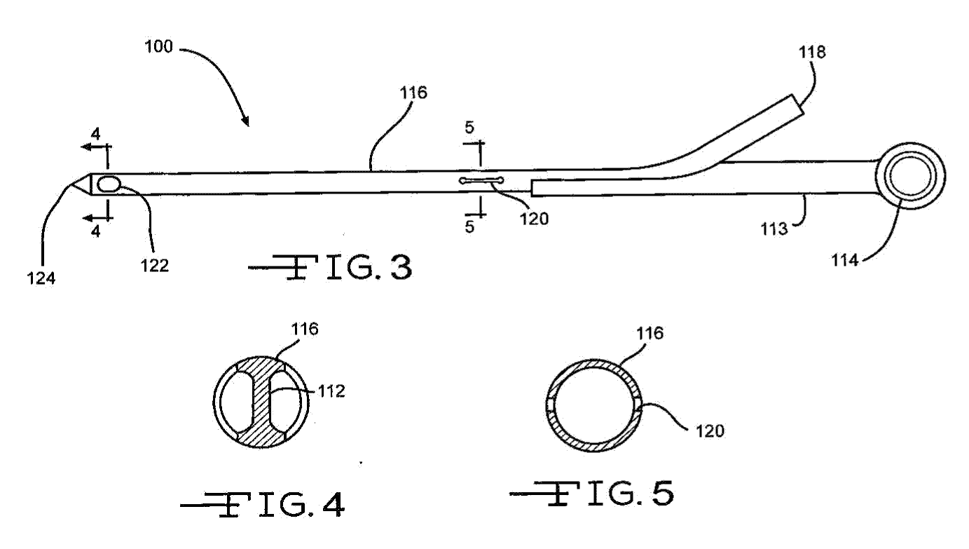

It should be noted this method also was effective means of physically harvesting marrow tissue, primarily fat and cells. An instrument 100, having a T-handle 112 at the proximal end and hollow tube 116 at the opposing distal end, was designed for such purpose (FIGS. 2-5). The instrument 100 comprises a hollow tube 116 having a solid closed pointed tip 124 at the distal end of the instrument 100 and an opposing open end 118 attached to a T-handle 112 with a shaft 113 and grip 114 for manipulation of the instrument 100. The tube 116 has distal openings 122 immediately adjacent to the tip which allow fat and cells exposed by the disruption of the bony architecture to enter the tube 116. There are secondary slits 120 along the tube 116 for two purposes. One is to provide a secondary vent to avoid clogging of the distal openings 122 due to the high suction causing occlusion. In addition, as the instrument is advanced the secondary slits 120 serve as a secondary method to harvest liquid fat and cells. The marrow material is drawn out of the tube and collected in a sterile canister for future use. The present invention encompasses instruments of like kind which can have larger openings to get more specimen and especially cancellous bone for BMP (Bone Morphogenetic Protein) future use. The stem cells, progenitor cells and/or macrophages which are isolated can be used in a treatment in combination with the bone surgery, or saved for long term by freezing.

The second intra operative method to be proposed is the use of an venous line for real time apheresis. This method would harvest the marrow cells that are mobilized during implantation of the instrumentation or implant. The cells would be cared for in same or similar manner following customary apheresis.

It should be noted that both of these intra operative methods provide routine access to marrow cells while providing prophylaxis for potential intraoperative complication of fat embolism. These methods require minimal additional time, equipment and expense. The potential twofold benefits are enormous. This intra operative method may provide the same potential for harvesting a person's stem cells for future use as does the well known method of saving frozen umbilical cord blood. The shelf life of the frozen cells is known to be at least 15 years.

The instrumentation for such harvesting can be value added for each and every such surgical procedure. The patient having the potential future use of autogenous stem cells.

A rod shaped instrument of greater the 6 millimeters in diameter introduced into the proximal femur will result in pushing marrow tissue out of the vascular channels of a femur. The tissue is mobilized through the vessels exiting the bone via the vascular channels. Stem cells, progenitor cells and/or macrophages can be directly retrieved at time of bone surgery. Typically this would be at time of exposing the marrow space in total joint surgery or open reduction internal fixation (ORIF) of long bones. The retrieval of marrow tissue and subsequently stem cells, progenitor cells and/or macrophages cells would be made at the time that the marrow cavity is exposed. An instrument could be placed down the canal with suction attached to retrieve the cells. There would be an added benefit in that the decompression of the marrow cavity would be intended to avoid or minimize pushing this tissue intra vascular during subsequent placement of the surgical implant.

Alternatively, stem cells, progenitor cells and/or macrophages cells can be indirectly retrieved from the circulating blood at time of surgical implantation. Bone marrow enters the bloodstream during intramedullary stem or rod placement. Blood can be drawn by venapunture at this time in the procedure to harvest the marrow material including the stem cells, progenitor cells and/or macrophage cells therein. Alternatively, the patient can be connected to a filtering apparatus similar to that portion of heart lung machine. Another method would be to use a cell retrieval devices such as those used in orthopedic surgery to save patients own blood extruded into the surgical wound to collect the stem cells, progenitor cells and/or macrophage cells.

Mobilization via Magnetic Resonance Imaging (MRI): Magnetic Resonance Imaging (MRI) requires exposing a patient to an external magnetic field and an applied oscillating electromagnetic field. Since MRI gives very clear images of tissues near bones, it is often used for diagnosis of joint injuries, arthritis, and herniated disks. It is also useful for osteomyelitis infections and tumors in bone and joints. In this manner the MRI diagnostic modality can be used to expose bone with the magnetic fields and radiofrequency pulses to mobilize stem cells, progenitor cells and/or macrophages into the bloodstream where they can be harvested incidental to an MRI scan. Alternatively an external magnetic field and an applied oscillating electromagnetic field can be applied having field strengths and durations optimized specifically for the purpose of mobilizing stem cells, progenitor cells and/or macrophages which are then isolated as described herein.

Mobilization via Ultrasound Exposure: Ultrasound and/or shockwave exposure can be used to mobilize stem cells, progenitor cells and/or macrophages. Ultrasound of the optimal intensity and duration can be executed intentionally for the purpose of mobilizing stem cells, progenitor cells and/or macrophages and collected as described herein. Alternatively the cells can be isolated during or after lithotripsy treatments. Lithotripsy is common medical procedure for the breaking up of renal and gall stones. Shock wave energy applied at the focal point is defined as the energy flux density (EFD) per impulse, with units of joules per area (mJ/mm²). The total energy of treatment is calculated using the number and EFD of each single impulse and the geometric focal area. Low-energy shock waves are defined as having an EFD of less than 0.1 mL/mm², while high-energy shock waves are defined as having an EFD of 0.2 to 0.4 mJ/mm². High-energy shock waves are capable of fragmenting bones and cartilage. Lithotripsy is one procedure encompassed by the present invention for mobilizing stem cells, progenitor cells and/or macrophages. Blood can be collected at appropriate times after exposure to harvest the cells. Other medical uses of ultrasound including diagnostic or therapeutic procedures are also encompassed by the present invention for the mobilization of stem cells, progenitor cells and/or macrophages and collected as described herein.

Cell Harvesting: Originally stem and progenitor cells were harvested by bone marrow biopsy or aspiration. This surgical procedure is often performed within a hospital and has an accompanying morbidity. Mesenchymal stem cells have been harvested from marrow, periosteum and muscle connective tissue. Recently, stem cells have been identified outside of the marrow in a variety of tissues including fatty tissue and in the circulating blood. This discovery lead to the advent of two chemical substances that can be injected into the patient and increase the yield of progenitor cells in the peripheral blood. Stem cell quantities obtained from the apheresis device are low and often require a number of days to remove sufficient volumes of blood for transplantation procedures depending upon the situation. Most hospitals perform leukophoresis, a method of separating out patient cells from blood. For any engraftment procedure, the number or stem and progenitor cells recovered from the bone marrow must be known. The number of blood progenitor cells can be measured by the colony forming unit-granulocyte macrophage (CFU-GM) assay, however the assay takes ten to fourteen days to complete, which is too slow for clinical relevance. The CD34 antigen is a useful indicator for measuring the potential for engraftment. CD34 is an adhesion molecule which is expressed on only a few percent of primitive bone marrow cells. The CD34 antigen is associated with human hematopoietic progenitor cells. It is found on immature precursor cells and all hematopoietic colony-forming cells, such as CFU-GM and BFU-E unipotent cells, and CFU-GEMM, CFU-Mix, and CFU-Blast pluripotent progenitors. Fully differentiated hematopoietic cells lack the CD34 antigen. Almost all of the colony-forming unit activity is found in CD34 expressing populations in human bone marrow.

CD34 antigen has been widely used to estimate the number of stem cells in a cell population and to enrich for stem cell populations. The CD34 anitigen is an approximately 110-115 kilodalton monomeric cell surface glycoprotein that is expressed selectively on human hematopoietic progenitor cells. The partial amino acid of a highly purified CD34 antigen has been analyzed, and it was found that it had no significant sequence similarity with any previously described structures. The antigen is not a leukosialin/sialophorin family despite structural similarities, and from a cDNA clone for CD34 from a KG-1 cell library enriched using the anti-CD34 monoclonal antibodies MY10 and BI-3C5 it has been determined to be a sialomucin. Hematopoietic cell lines KG-1, KMT-2, AML-1, RPMI 8402, and MOLT 13 express a 2.7 kilobase CD34 transcript. The cDNA sequence codes for a 40 kilodalton type I integral membrane protein with nine potential N-linked and many potential O-linked glycosylation sites which is a type I transmembrane protein. The 28 kilobase CD34 gene includes eight exons mapped from the coding sequences. The CD34 transcription start site is 258 base pairs upstream of the start site of translation. Anti-CD34 monoclonal antibodies My10 and 8G12, known in the art, bind to two different epitopes of the CD34 antigen expressed on stem cells. Lineage-specific antigens CD71, CD33, CD10, and CD5 are lacking on progenitor cells which are not lineage committed (CD34+CD38−). The CD34 antigen can be used to estimate stem cell enrichment. It is estimated that a minimum of approximately 2.5×10⁶ CD34⁺ progenitors per kilogram patient weight are needed for effective hematopoietic reconstitution during bone marrow transplantation procedures.

Populations of stem cells and progenitor cells from the peripheral bloodstream can be enriched by utilizing surface markers such as c-kit, CD34 , and H-2K. Surface markers such as Lin are typically lacking, or expressed at very low levels, in stem cells, so Lin can be a negative selection marker. Cells that are CD34+ Thy1+lin−. Sca-1 expressing lineage depleted (lin neg). Cell-surface antigens which can be used to positively or negatively select for undifferentiated hematopoietic stem cells include, but are not limited to, CD34+, CD59+, Thy1+, CD38(low/−), C-kit (−/low), lin−. Positive selection of marrow for CD34+CD33− hematopoietic progenitors, and use of c-kit ligand can be used for ex-vivo expansion of early hematopoietic progenitors.

Stem cells have also been isolated by density-gradient centrifugation from bone marrow aspirates. Mesenchymal stem cells have been shown to adhere to polystyrene while other cells found in bone marrow aspirates, i.e. cells of hematopoietic lineage do not adhere to polystyrene tissue culture materials.

Recently it has been discovered that hematopoietic stem cells, which are derived from mesoderm, can give rise to skeletal muscle, which is derived from mesoderm, and neurons, derived from ectoderm. This capability has been termed “plasticity”, “unorthodox differentiation”, or “transdifferentiation” in the literature. In one embodiment of the present invention, the stem cells are used to repair or regenerate skeletal muscle. In another embodiment of the present invention the stem cells are used to repair or regenerate neural tissue.

Recently, cancer stem cells have been isolated from certain cancers. In another embodiment of the present invention cancer stem cells are isolated for further study.

Once the stem cells, pluripotential cells progenitor cells, or macrophages have been harvested, an aliquot can be taken to grow out so as to identify and prove that they are of the desired cell type by culturing procedures and assays known in the art. The stem cells, pluripotential cells or progenitor cells can be frozen and stored by protocols known in the art and described in U.S. Pat. Nos. 5,004,681; 5,192,553; 6,461,645; 6,569,427 and 6,605,275 to Boyse et al. incorporated herein by reference in their entirety.

Cell introduction: It has been postulated that circulating marrow progenitor cells find their way to the local areas of injury for healing influence. The stem cells have the capacity to home in on specific tissues and engraft within the tissue. The process is not thoroughly understood, however various adhesion receptors and ligands which mediate the cell-matrix and cell-cell binding have been studied (Quesenberry and Becker, Proc. Natl. Acad. Sci. USA, vol. 95, pp. 15155-15157 (1998)). Some of the adhesion molecules studied include L, P and E selecting, integrins, VCAM-1, ICAM-1, VLA-4, VLA-5, VLA-6, PECAM, and CD44. The stem cells can therefore be infused via a large-bore central venous catheter, whereupon the stem cells will home in to the tissue in need of repair. Alternatively, the stem cells can be surgically implanted at a specific site. Allogenic transplants require careful donor and recipient matching for major histocompatibility (HLA) antigens. In the case of hematopoietic stem cell transplantation for bone marrow reconstitution graft-versus-host disease (GVHD) must be considered. Alternatively, since it is known that blood cells collect at wounds, and that circulating white blood cells selectively travel to the wound and participate in wound healing, any of the physical means can be applied to the patient to mobilize stem cells, progenitor cells and/or macrophages to enhance healing of wounds in the patient. It has recently been discovered that fetal CD34+ cells enter the maternal bloodstream and persist for decades and may develop multilineage capacity in maternal organs (JAMA Jul. 7, 2004; 292(1): 75-80).

Clinical applications of the present invention include methods to retrieve cells in any general hospital whereupon the cells will be readily available for transplant for cancers including leukemia, and cartilage and/or bone injury and diseases. Indications for allogeneic hematopoietic stem cell transplants include: acute leukemia, myelodysplastic syndrome, chronic myeloid leukemia, severe aplastic anemia, indolent lymphoma, chronic lymphocytic leukemia, severe immunodeficiency syndromes, and hemoglobinopathies. Indications for autologous hematopoietic stem cell transplantation include: progressive large-cell lymphoma, progressive Hodgkin's disease, multiple myeloma, relapsed germ cell tumors. The present invention can be used to repair or regenerate bone marrow for the treatment of these cancers. In other embodiments the invention can be used to repair or regenerate other tissues, including but not limited to organs, cartilage, bone and spinal cord injury. Cardiac muscle can by treated as described in U.S. Pat. No. 6,387,369 to Pittenger et al., hereby incorporated herein by reference in its entirety. Connective tissue can by treated as described in U.S. Pat. Nos. 5,197,985; 5,226,914 and 5,811,094 to Caplan et al., hereby incorporated herein by reference in their entirety. Chondrogenesis can be promoted as described in U.S. Pat. No. 5,908,784 to Johnstone et al., hereby incorporated herein by reference in its entirety. The cells can be implanted into the damaged tissue using a matrix such as described in U.S. Pat. No. 6,174,333 to Kadiyala et al. Research is being done on the application of stem cells for a wide array of uses (Eg. Scheffold et al., Purified allogeneic hematopoietic stem cell transplantation prevents autoimmune diabetes and induces tolerance to donor matched islets. Blood. 1999;94 (suppl 1): 664a.; Perry T. E. and Roth S. J., Cardiovascular tissue engineering: constructing living tissue cardiac valves and blood vessels using bone marrow, umbilical cord blood, and peripheral blood cells. J. Cardiovasc Nurs. 2003;18:30-37.)

Spinal cord injury is now being investigated with culture of macrophages retrieved from blood, cultured and injected in and around cord injury within two weeks which results in decreased inflammation. Early clinical results indicate that the treatment can be motor and sensory sparing. The present invention encompasses mobilizing and collecting macrophages and/or their precursors, such as monocytes, for therapeutic purposes. Spinal cord injury patients often have fractured femurs and other long bone fractures which allow access to the bone marrow cells. BMP, bone morphogenic protein is being used in number of ways for healing of fractures and cartilage healing. The bone marrow harvested during surgery is a potential source of this protein, which would be autogenous. I have published on the use of cancellous bone from marrow. There also is in the literature the use of needle aspiration of marrow and subsequent injection along fractures that are not healing to speeding the healing process. Therefore, cancellous bone harvested at open surgery of total joint or fracture can be used for assisting the healing of nonunions of fractures and also has potential for prophylaxis in fracture treatment. People who get total hip replacements often have fracture complications at or after surgery below the implant. Having their marrow would be great potential adjunct to a minimally invasive means of treatment.

For testing, animals can be used in an operating room with a good leg dropped into mini lithotripsy bath and blood harvested. Localized lithotripsy on iliac crest spread progenitor cells into the blood stream whereupon the cells can find their way to the localized injury and promote healing. The method encompasses lithotripsy, general or localized to a limb, axial skeleton and other ultrasound treatments to bone. Specific temperatures of the lithotripsy bath fluid, wave lengths, timing of ultra sound application, the optimal coordinated time of harvest, and the optimal amount of cell volume for optimal treatment to bone and/or cartilage are encompassed by the present invention. In one embodiment, the iliac crest 11 of a human patient 10 is exposed to shock waves generated by a lithotripter 20. Peripheral blood cells are collected using a dual lumen catheter 31 and pass to a leukaphoresis device 30 via a collection line 32, where cells are separated and remaining blood components are returned to the patient 10 through return line 33. Similarly setups can be used in MRI and CAT scan embodiments of the present invention.

### EXAMPLES

The mobilization of stem cells, progenitor cells and/or macrophages in patients undergoing lithotripsy, and animals undergoing ultrasound to the sternum or pelvis are tested. Patients undergoing lithotripsy agree to blood analysis before and immediately after lithotripsy as well as one hour and one day later. Peripheral blood is removed and analyzed for stem cells, progenitor cells and/or macrophages by any test known in the art and the two samples are compared. Animal studies are performed to confirm the value of local application and then the ultrasound, increase blood cells finding the local lesions for healing. Mobilization of cells by means of MRI or CAT scans can also be tested in like manner.

It is proposed that certain constructs of ultrasound instrumentation, frequency, pulse intervals and dose will cause reconversion of the bone marrow. Certain ultrasound therapy will cause the fatty yellow bone marrow as seen in elderly to in medical terms to undergo reconversion to the vascular cellular red bone marrow seen in youth. This may be considered rejuvenation. The potential benefits would be a bone marrow productive of stem cells and hematopoietic elements with immune factors that may not only prolong life, but enhance the quality of life.

Recoversion of Bone Marrow by UltraSound Therapy: The medical literature supports the concept of changing the vascularity and cellularity of tissues subject to various both low level and focused high intensity ultrasound. The efficacy is established in that it does happen in all tissues subject to the therapy to date in laboratory and/or clinical trials, except bone, not to be confused with bone marrow which is housed inside the bone and harbors the stem cells and hematopoietic system.

Mechanism of action of Ultrasound: The mechanism of action is due to heat. The tissue response is probably due to growth factors.

Ultrasound-biophysics is the study of how ultrasound and biological materials interact. Ultrasound-induced bioeffects are generally separated into thermal and non-thermal mechanisms. Ultrasonic dosimetry is concerned with the quantitative determination of ultrasonic energy interaction with biological materials. Whenever ultrasonic energy is propagated into an attenuating material such as tissue, the amplitude of the wave decreases with distance. This attenuation is due to either absorption or scattering. Absorption is a mechanism that represents that portion of ultrasonic wave that is converted into heat, and scattering can be thought of as that portion of the wave, which changes direction. O'Brien W D Jr, Mechanism of action of ultrasound Pro Biophys Mol Biol Aug. 8, 2006

The release of growth factors have been identified following ultrasound treatment. “The mechanism of shock wave therapy involved the early release of angiogenic growth factors (eNOS and VEGF) and subsequent induction of neovascularization and tissue proliferation. The neovascularizatoin may play a role in pain relief of tendonitis and the repair of chronically inflamed tendon tissues at the tendon-bone junction.” Wang C. Shock Wave Therapy Induces Neovascularization at the Tendon-Bone Junction: A Study in Rabbits Journal of Orthopaedic Research, 21 (2003) pp. 984-989

Treatment of Osteonecrosis (ON) of the Femoral Head: ON is literally death (necrosis) of the bone due to lack of a local blood supply. Existing treatment methods are not efficient or predictable. However, recent publications have shown effective treatment with high density focused ultrasound. Extracorporeal shock wave treatment appeared to be more effective than core decompression and nonvascularized fibular grafting for providing short-term pain relief for patients affected by early stages of osteonecrosis of the femoral head. Wang C, et al. J Bone Joint Surg 2005

Histological studies suggest that low intensity pulsed ultrasound stimulation (LIPUS) influences all major cell types involved in bone healing, including osteoblasts, osteoclasts, chondrocytes and mesenchymal stem cells. The affect of LIPUS seems to be limited to cells in soft tissue, whereas cells in calcified bone seem not to be effected. The most probable source of the therapeutic benefits observed with LIPUS treatment involves non-thermal mechanisms that influence cell membrane permeability and increase cellular activity. Claes L et al Prog Biophys Mol Biol, Aug. 10, 2006

Histological evidence shows exams at 4 and 16 weeks after ESWT found increased tenocyte production with neovascularization at 16 weeks. Hsu R. .Effect of ESWT on tendon pathology in Rabbit Model. Journal of Orthopaedic Research, 22 (2004) pp. 221-227

There is evidence that ischemic extremity and myrocardial vascular perfusion is increased by certain doses of ultrasound. Am Coll Cardiol. Oct. 6, 2004;44:1454-8

Ultrasound therapy promotes neovascularization in tissues and organs. It is the pathophysiological basis of clinical response of the myocardium as mentioned above. It is the histological basis for positive clinical response to fracture healing, tennis elbow, plantar fasciitis, calcific tendonitis of the shoulder. There is both laboratory animal and clinical evidence in the literature. Wang found that “the mechanism of shock wave therapy involved the early release of angiogenic growth factors (eNOS and VEGF) and subsequent induction of neovascularization and tissue proliferation. The neovascularizatoin may play a role in pain relief of tendonitis and the repair of chronically inflamed tendon tissues at the tendon-bone junction.”
Wang C J, Huang H Y, Pai C H. Shock wave-enhanced neovascularization at the tendon-bone junction: An experiment in dogs. J Foot Ankle Surg. 2002;41(1):16-22.
Wang C, Want F S, Yang K D, Weng L H, Hsu C C, Huang C S, Yang L C. Shock wave therapy induces neovascularization at the tendon-bone junction. A study in rabbits. J Ortho Res. 2006.21(6), 984-989.

Bone Healing: Low Intensity ultrasound has been used for healing of fracture non unions. The most probable source of the therapeutic benefits observed with LIPUS treatment involves nonthermal mechanisms that influence cell membrane permeability and increase cellular activity. In vitro cell culture studies as well as tissue culture studies have shown some effects on cell differentiation and protein synthesis. Even though the energy used by LIPUS treatment is extremely low, the effects are evident. Despite clinical and experimental studies demonstrating the enhancing effect of LIPUS on bone regeneration, the biophysical mechanisms involved in the complex fracture healing process remain unclear and requires further research. Claes, L et al The enhancement of bone regeneration by ultrasound Prog Biophys Mol Biol. Aug. 10, 2006

Calcific Tendonitis of Shoulder Tendons: Additional support for revasculariztion of tissue by ultrasound is found in the treatment of calcific tendonitis of the shoulder. This condition has a collection of a necrotic tissue with paste like consistency with calcium and absence of blood vessels. The traditional method of treatment was multiple needle puncture and aspiration if possible to promote revascularization. Ultrasound has now been used for the same purpose based upon promoting revascularization.
Harniman E, Carette S, Kennedy C, Beaton D. Extracorporeal shock wave therapy for calcific and noncalcific tendonitis of the rotator cuff: A systematic review. J Hand Ther. 2004;17(2):132-151.
Noel E, Charrin J. Extracorporeal shock wave therapy in calcific tendinitis of the shoulder. Rev Rhum Engl ed. 1999;66(12):691-693.
Loew M, Daecke W, Kusnierczak D, et al. Shock-wave therapy is effective for chronic calcifying tendinitis of the shoulder. J Bone Joint Surg Br. 1999;81(5):863-867.
Rompe J D, Burger R, Hopf C, Eysel P. Shoulder function after extracorporal shock wave therapy for calcific tendinitis. J Shoulder Elbow Surg. 1998;7(5):505-509.
   Plantar Fasciitis
Kudo P, Dainty K, Clarfield M, et al. A randomized, placebo-controlled, double-blind clinical trial evaluating the treatment of plantar fasciitis with an extracorporeal shockwave therapy (ESWT) device; A North American confirmatory study. J Orthopaed Res. 2006;24:115-123.
Theodore G H, Buch M, Amendola A, et al. Extracorporeal shock wave therapy for the treatment of plantar fasciitis. Foot Ankle Int. 2004;25(5):290-297.
Boddeker I R, Schafer H, Haake M. Extracorporeal shockwave therapy (ESWT) in the treatment of plantar fasciitis: A biometrical review. Clin Rheumatol. 2001;20(5):324-330.
Haake M, Buch M, Schoellner C, et al. Extracorporeal shock wave therapy for plantar fasciitis: Randomised controlled multicentre trial. BMJ. 2003;327(7406):75.
Wang C J, Chen H S, Huang T W. Shockwave therapy for patients with plantar fasciitis: A one-year follow-up study. Foot Ankle Int. 2002;23(3):204-207.
Speed C A, Nichols D W, Wies J, et al. Extracorporeal shock wave therapy for plantar fasciitis. A double blind randomised controlled trial. J Orthop Res. 2003;21(5):937-940.
Kudo P, Dainty K, Clarifield M, Coughlin L, Lavoie P, Lebrun C. Randomized, placebo-controlled, double-blind clinical trial evaluating the treatment of plantar fasciitis with an extracorporeal shockwave therapy (ESWT) device: A North American confirmatory study. Journal of Ortho Res 2006, vol. 24(2),115-123.
Lateral Epicondylitis
Cosentino R, De Stefano R, Selvi E, et al. Extracorporeal shock wave therapy for chronic calcific tendinitis of the shoulder: Single blind study. Ann Rheum Dis. 2003;62(3):248-250.
Rompe J D, Hopf C, Kullmer K, et al. Analgesic effect of extracorporeal shock wave therapy on chronic tennis elbow. J Bone Joint Surg. 1996;78-B(2):233-237.
Haake M, Konig I R, Decker T, et al. Extracorporeal shock wave therapy in the treatment of lateral epicondylitis: A randomized multicenter trial. J Bone Joint Surg Am. 2002;84-A(11):1982-1991.
Speed C A, Nichols D, Richards C, et al. Extracorporeal shock wave therapy for lateral epicondylitis—a double blind randomised controlled trial. J Orthop Res. 2002;20(5):895-898.
Melikyan E Y, Shahin E, Miles J, Bainbridge L C. Extracorporeal shock-wave treatment for tennis elbow. A randomised double-blind study. J Bone Joint Surg Br. 2003;85(6):852-855.
Stasinopoulos D, Johnson M I. Effectiveness of extracorporeal shock wave therapy for tennis elbow (lateral epicondylitis). Br J Sports Med. 2005;39(3):132-136.

The FDA which judges efficacy and safety has approved various treatment modalities. There is approval of the treatment with low intensity ultrasound for lateral condylar tendonitis (Tennis elbow) and plantar fasciitis. There is substantial support in the literature for such treatments.

The FDA has approved the use of extracorporeal shockwave the treatment of multiple orthopedic conditions which have failed to respond to conservative treatment. (OssaTron® is commercial entity first approved) Wang reported 72 subjects with long bone nonunions were studied—40% had boney union at 3 months, 60.9% at 6 months and 80% at 12 months post-ESWT
Rompe J D, Rosendahl T, Schollner C, Theis C. High-energy extracorporeal shock wave treatment of nonunions. Clin Orthop. 2001;(387):102-111.
Birnbaum K, Wirtz D C, Siebert C H, Heller K D. Use of extracorporeal shock-wave therapy (ESWT) in the treatment of non-unions. A review of the literature. Arch Orthop Trauma Surg. 2002;122(6):324-330.
Biedermann R, Martin A, Handle G, et al. Extracorporeal shock waves in the treatment of nonunions. J Trauma. 2003;54(5):936-942.
Wang C. Treatment of Nonunions of Long Bone Fractures with Shock Waves. Clinical Orthopaedics and Related Research, No. 387, June 2001

The FDA has approved Ultrasonic osteogenesis stimulation (SAFHS) for healing of certain bone fracture conditions with known or anticipated slow healing.

When applied over a fracture site, the SAFHS device produces an ultrasonic wave, which delivers mechanical pressure to the bone tissue at the fracture site. Although the mechanism by which the low intensity pulsed ultrasound device accelerates bone healing is uncertain, it is thought to promote bone formation in a manner comparable to bone responses to mechanical stress.
Heckman J D, Ryaby J P, McCabe J, et al. Acceleration of tibial fracture-healing by non-invasive, low-intensity pulsed ultrasound. J Bone Joint Surg Am. 1994;76(1):26-34.
Kristiansen T K, Ryaby J P, McCabe J, et al. Accelerated healing of distal radial fractures with the use of specific, low-intensity ultrasound. A multicenter, prospective, randomized, double-blind, placebo-controlled study. J Bone Joint Surg Am. 1997;79 (7):961-973
Cook S D, Ryaby J P, McCabe J, et al. Acceleration of tibia and distal radius fracture healing in patients who smoke. Clin Orthop. 1997;337:198-207.
Hadjiargyrou M, McLeod K, Ryaby J P, et al. Enhancement of fracture healing by low intensity ultrasound. Clin Orthop. 1998;355 Suppl:S216-S229.
Scott G, King J B. A prospective, double-blind trial of electrical capacitive coupling in the treatment of non-union of long bones. J Bone Joint Surg. 1994;76A(6):820-826.

The safety of ultrasound treatment is supported by the fact that when normal bone is subject to known levels of ultrasound there is no deleterious effect on the hematopoietic system. Ultrasound promotes growth and differentiation of bone marrow cells (Efficacy) , but ESW treatment did not affect haematopoiesis.

### REFERENCES

Wang F S, Yang K D, Chen R F, Wang C J, Sheen-Chen S M. Extracorporeal shock waves promotes growth and differentiation of bone-marrow stromal cells towards osteoprogenitors associated with induction of TGF-(beta)l. J Bone Joint Surg 2002; 84: 457-461.

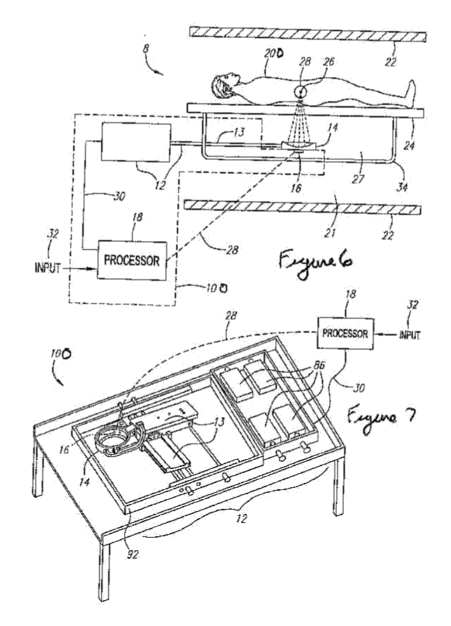

Turning to FIGS. 6 and 7, FIG. 6 illustrates an exemplary embodiment of a focused ultrasound system 8 including an ultrasonic transducer 14, a positioning system 100 for positioning the ultrasound transducer 14, and a magnetic resonance imaging (“MRI”) system 22. The positioning system 10 includes a positioner 12 coupled to the ultrasound transducer 14, a sensor 16 carried by the ultrasound transducer 14, and a processor 18 coupled to the positioner 12 and sensor 16.

The ultrasound transducer 14 may be mounted within a chamber 27 filled with degassed water or similar acoustically transmitting fluid. The chamber 27 may be located within a table 34 upon which a patient 200 may be disposed, or within a fluid-filled bag mounted on a movable arm that may be placed against a patient's body (not shown). The contact surface of the chamber 27, e.g., the top 24 of the table 34, generally includes a flexible membrane (not shown) that is substantially transparent to ultrasound, such as mylar, polyvinyl chloride (PVC), or other suitable plastic material. Optionally, a fluid-filled bag (not shown) may be provided on the membrane that may conform easily to the contours of the patient 200 disposed on the table, thereby acoustically coupling the patient 200 to the ultrasound ultrasound transducer 14 within the chamber 27. In addition or alternatively, acoustic gel, water, or other fluid may be provided between the patient 200 and the membrane to facilitate further acoustic coupling between the transducer 14 and the patient 200.

In addition, the transducer 14 may be used in conjunction with an imaging system. For example, the table 34 may be positioned within an imaging volume 21 of an MRI system 22, such as that disclosed in U.S. Pat. Nos. 5,247,935, 5,291,890, 5,368,031, 5,368,032, 5,443,068 issued to Cline et al., and U.S. Pat. Nos. 5,307,812, 5,323,779, 5,327,884 issued to Hardy et al., the disclosures of which are expressly incorporated herein by reference.

In order to position the ultrasound transducer 14, e.g., to direct a focal zone 26 of the transducer 14 towards a target bone marrow region 28 within the patient 200, the positioner 12 may move the ultrasound transducer 14 in one or more degrees of freedom. For example, the transducer 14 may be rotated, or translated relative to the patient 200. The positioner 12 is typically distanced away from the MRI system 22, e.g., outside the imaging volume 21 in order to minimize interference. Known positioners, which may include one or more motors, drive shafts, joints, and the like, have been described in U.S. Pat. Nos. 5,443,068, 5,275,165, and 5,247,935, and in the U.S. patent application Ser. No. 09/628,964, the disclosures of which are expressly incorporated by reference herein.

FIG. 7 illustrates a system 100 for positioning the ultrasound transducer 14 according to a preferred embodiment. As used here, positioning includes translating or moving the ultrasound transducer 14 to a new location in space, as well as rotating or tilting the transducer 14 about an axis to achieve a new orientation of the transducer 14. The positioner 12 shown in FIG. 7 may provide roll and pitch control of the transducer 14, as well as lateral and longitudinal control. The positioner 12 may include piezoelectric vibrational motors 86 that may operate within the field of an MRI system without interfering substantially with its operation, such as those described in U.S. patent application Ser. No. 09/628,964, filed Jul. 31, 2000, which is incorporated by reference herein. The motors 86 may provide a braking force to the drive shafts (not shown) while de-energized and thus aid in preventing motor slippage or backlash. The positioner 12 may also include a set of encoders (not shown), which are described in the U.S. patent application Ser. No. 09/628,964, coupled to the positioning motors 86 to control the position of the transducer 14.

Returning to FIG. 6, the processor 18 may include one or more logic circuits, a microprocessor, and/or computers coupled to the sensor 16 to receive signals from the sensor 16, and to the positioner 12 for directing the positioner 12 to move the ultrasound transducer 14 in a translational or rotational motion. The processor 18 may be a separate subsystem from a controller or other subsystems (not shown) used to operate the ultrasound transducer 14 and/or the MRI system 22. Alternatively, the processor 18 may be included in a computer that includes hardware components and/or software modules for performing other functions of the system 8, e.g., controlling the ultrasound transducer 14 and/or the MRI system 22.

A first communication path 28 allowing signals to be communicated from the sensor 16 to the processor 18 may include one or more wires coupling the sensor 16 to the processor 18. In addition or alternatively, the first communication path 28 may include an optical cable and/or a wireless transmitter for transmitting signals from the sensor 16 to the processor 18. A wireless transmitter may transmit signals, such as radio frequency, infrared, or other signals, to a receiver (not shown) coupled to the processor 18. The frequency of such radio frequency signals may be selected to minimize interference with the MRI system. Similarly, the second communication path 30, which couples the processor 18 and the positioner 12, may include one or more wires, optical cables, and/or a wireless transmitter.

The positioning system 100 may also include an interface, such as a keyboard, a mouse, and/or touch screen (not shown) for providing an input 32 to the processor 18, the positioner 12, and/or other components of the system 8, as described below.

To use the system 100, a user may enter an input 32, preferably through the interface, which may define or otherwise include a desired position of the transducer 14 for example the bone marrow of the leg. As used herein, “position” may include one or both of a location in space (e.g., in one, two, or three dimensions) and an orientation (e.g., a pitch or roll angle) of the transducer 14. Preferably, the desired position of the transducer 14 includes a translation location along the predetermined axes and/or a rotational orientation of the transducer 14 about determined axes.

Once the processor 18 receives an input 32 identifying a desired position of the ultrasound transducer 14, the processor 18 may transmit a signal to the positioner, instructing the positioner 12 to move the ultrasound transducer 14 based at least in part on the input 32 to the desired position. For example, the processor 18 may instruct the positioner 12 to move the ultrasound transducer 14 based upon a calculation performed by the processor 18, e.g., a difference between the desired position and a current position of the transducer 14.

Alternatively, the positioner 12 may receive the input 32 directly and may move the transducer 14 based at least in part on the input 32. In this alternative, the input 32 (or the desired position) may be transmitted from the positioner 12 to the processor 18.

Once the positioner 12 has moved the transducer 14, the sensor 16 may measure an actual position of the transducer 14 and compare it to the desired position. For example, the processor 18 may receive one or more data signals from the sensor 16, e.g., via the first communication path 28. The processor 18 may then determine the true tilt angle based on the sensor measurement and, optionally, a set of calibration coefficients. The calibration coefficients may be associated with coordinate transformation, as is known in the art, which relates the mounting position of the sensor 16 to the coordinate system of the transducer 14. In particular, the calibration coefficients may be used to correct misalignment between the coordinate systems of the transducer 14 and the sensor 16, and to account for the geometric relation between the sensor's measurement axis and the transducer rotation axis. The calibration coefficients may be initially or periodically determined using a calibration procedure, such as that discussed below.

If the true position of the ultrasound transducer 14 does not match the desired position, the processor 18 may direct the positioner 12 to adjust the position of the transducer 14, for example, based on the difference between the true position and the desired position. This iterative process of obtaining the position data, determining the true position, comparing the true and desired positions, and adjusting the position of the ultrasonic transducer 14, may be repeated until the desired position associated with the user's input 32 is achieved within an acceptable tolerance level. For example, the desired tilt angle may be considered to be achieved if the true tilt angle is within a predetermined range around the desired tilt angle, such as within 0.25 degree of the desired tilt angle.

Once the ultrasound transducer 14 is in the desired position, the ultrasound transducer 14 is activated to generate ultrasound waves directed to the selected bone marrow. The ultrasound waves result in the generation of stem cell, progenitor cells and/or macrophages which in turn repairs the damaged tissue. That limitation, the damaged tissue may be repaired by increasing the cellular of the bone marrow. In further embodiments of the method and system, the fatty bone marrow is reduced. In further embodiments of the method and system, the vascular of the bone marrow is increased.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the Claims attached herein.

1. A method for treating a patient to repair damaged tissue which comprises:
(a) exposing a selected area of bone marrow of a patient to ultrasound waves or ultra shock waves so that cells comprising stem cells, progenitor cells or macrophages are generated in the area of the bone marrow of the patient due to the ultrasound;
(b) converting the cells from the bone marrow of the patient; and
(c) reducing the damaged tissue in the bone marrow of the patient by repairing the damaged tissue.

2. The method of claim 1 wherein the area comprises the bone marrow in a trunk or extremity of the patient.

3. The method of claim 1 wherein the area comprises the bone marrow in the rib of the patient.

4. The method of claim 1 wherein the area comprises the bone marrow in a hip of the patient.

5. The method of claim 1 wherein the area comprises the bone marrow in a shoulder of the patient.

6. The method of claim 1 wherein the area comprises the bone marrow in a back of the patient.

7. The method of any one of claim 1 wherein the patient is a human.

8. The method of any one of claim 1 wherein the patient is an animal.

9. The method of claim 1 wherein the area comprises the bone marrow of the head of the patient.

10. The method of claim 7 wherein the area is the bone marrow in an extremity of the patient.

11. The method of claim 7 wherein the bone marrow is in an arm or a leg.

12. The method of claim 7 wherein the area is the bone marrow in a trunk of the patient.

13. The method of claim 12 wherein the bone marrow is a sternum or an iliac crest.

14. A system for activating stem cells, pluripotential cells, progenitor cells or macrophages which comprises:
(a) a device which provides ultrasound waves or shock waves to an area of bone marrow of a patient so as to generate cells comprising stem cells, pluripotent cells, progenitor cells, macrophages and mixtures thereof in the bone marrow;
(b) a converter device for converting the cells of the bone marrow of the patient to reduce the damaged tissue in the bone marrow of the patient.

15. The system of claim 16 further comprising a fluid for transmitting the ultrasound waves.

16. The system of claim 16 wherein the bath is for an arm or a leg.

17. A method for treating a patient to repair damaged tissue which comprises:
(a) selecting a area in the bone marrow of the patient to be exposed;
(b) exposing the selected area in the bone marrow of the patient to a physical treatment of the body selected from the group consisting of ultrasound waves, ultra shock waves, so that cells comprising stem cells, progenitor cells or macrophages are generated in the bone marrow of the patient such that the stem cells, progenitor cells or macrophages repair the damaged tissue in the area in the bone marrow of the patient so as to repair the damaged tissue.

20. The method of claim 1 wherein the damaged tissue is repaired by increasing the cellular of the bone mar-row.

21. The method of claim 1 wherein the damaged tissue is repaired by reducing the fatty bone marrow.

22. The method of claim 1 wherein the damaged tissue is repaired by increasing the vascular of the bone marrow.
